(19) United States
(12) Patent Application Publication
(10) Pub. No.: US 2024/0412838 A1
(43) Pub. Date: Dec. 12, 2024

(54) KNOWLEDGE GRAPH-BASED METHOD FOR RECOMMENDING TRADITIONAL CHINESE MEDICINE PRESCRIPTIONS

(71) Applicant: HENAN UNIVERSITY OF SCIENCE AND TECHNOLOGY, Luoyang City (CN)

(72) Inventors: Mingchuan Zhang, Luoyang City (CN); Lin Wang, Luoyang City (CN); Qingtao Wu, Luoyang City (CN); Wenxuan Xu, Luoyang City (CN); Junlong Zhu, Luoyang City (CN); Xuhui Zhao, Luoyang City (CN); Muhua Liu, Luoyang City (CN); Ruijuan Zheng, Luoyang City (CN); Zhihang Ji, Luoyang City (CN); Moli Zhang, Luoyang City (CN)

(21) Appl. No.: 18/667,299

(22) Filed: May 17, 2024

(30) Foreign Application Priority Data
Jun. 12, 2023 (CN) 202310690324.1

(51) Int. Cl. G16H 20/10; G06N 3/0464; G06N 3/08

(52) U.S. Cl. CPC G16H 20/10; G06N 3/0464; G06N 3/08

(57) ABSTRACT

A knowledge graph-based method for recommending traditional Chinese medicine (TCM) prescriptions is provided. The method integrates a knowledge graph embedding model, a multi-head attention mechanism, graph convolutions, and other techniques to combine graph features with a recommendation system, for comprehensively considering patient conditions to recommend TCM prescription. Taking TCM case description texts as the research object and integrating with TCM knowledge graph information, the method draws from the clinical experience of renowned TCM experts and fully considers various individual factors such as medicinal properties, efficacy, medical conditions, and patient constitutions. Based on the holistic principles of TCM and the ideology of syndrome differentiation and treatment, the method selects different herbal combinations based on varying symptoms and medical conditions of patients. The proposed TCM prescription recommendation method, incorporating the knowledge graph, considers the complex relationships between individual signs as well as symptoms and medications.

### CROSS REFERENCE TO RELATED APPLICATION

This patent application claims the benefit and priority of Chinese Patent Application No. 202310690324.1 filed with the China National Intellectual Property Administration on Jun. 12, 2023, the disclosure of which is incorporated by reference herein in its entirety as part of the present application.

### TECHNICAL FIELD

The present disclosure relates to the field of traditional Chinese medicine (TCM) prescription recommendations, and in particular to a knowledge graph-based method for recommending TCM prescriptions.

### BACKGROUND

Traditional Chinese medicine (TCM) originated in ancient China and is a medical theoretical system that is formed through practice and summarization for thousands of years. It is a valuable asset and the crystallization of wisdom for the Chinese nation. In recent years, the Chinese government has attached great importance to the development and inheritance of TCM, actively taking measures to promote its dissemination and application both domestically and internationally, further promoting the inheritance, open innovation and development of TCM. TCM, as a unique medical system, uses various methods such as looking, asking, listening and smelling and touching to obtain disease and symptom information and then carry out treatment based on syndrome differentiation. In clinical TCM practice, it is necessary that appropriate prescriptions are selected based on a scientific differential diagnosis foundation, according to individual patient characteristics, and in compliance with the principles of herbal medicine compatibility. Unlike the simple addition of the individual herbs, the efficacy of TCM prescriptions is achieved through the comprehensive actions of the herbs to harmonize the balance of Yin and Yang in the human body, leading towards recovery from diseases.

However, currently, there is a lack of clear differentiation between the primary and secondary symptoms in TCM diagnosis and treatment, prescriptions are often not appropriate for actual symptoms, and the compatibility of herbal medicines is disorderly. The absence of a standardized knowledge mode for diagnosis and treatment based on classical TCM works and famous prescriptions. The lack of experience among primary healthcare physicians often results in less objective treatment and prescriptions due to subjective factors. Currently, TCM knowledge is scattered throughout numerous ancient texts and literature, and its loose and unstructured nature results in low utilization of TCM knowledge. Additionally, TCM takes a holistic approach, considering various factors such as the body, age, gender, and medical history of the patient, rather than just treating individual symptoms or diseases. Some studies have used attention-based neural networks to detect different herbal medicine groupings in TCM prescriptions, but such approaches often lack consideration for personalization. Furthermore, due to the potential side effects of herbal medicines, it is necessary to make a proper combination of herbs to achieve a balance in medicine effects and prevent the medicine effects from canceling each other or generating side effects. However, some research, such as the use of Bidirectional Recurrent Neural Networks (BRNN), for learning herbal medicine representations, has shown limited effectiveness in handling herbal medicine compatibility. These issues limit the application and development of TCM.

The application of a TCM knowledge graph can significantly enhance the dissemination of TCM knowledge. By integrating TCM knowledge graphs with artificial intelligence algorithms, it is possible to restructure the complex semantic relationships within the TCM theoretical system and uncover underlying associations, thereby making the TCM theory more scientific and standardized. Intelligent-assisted diagnostic and treatment systems can utilize TCM knowledge graphs to semantically infer the content of medical records, providing doctors with the most relevant diagnostic evidence and treatment options for patients. This, in turn, improves the efficiency of the doctors, reduces misdiagnoses and missed diagnoses, and offers personalized diagnostic and treatment plans for patients to enhance the quality of patient care. Therefore, TCM-assisted diagnosis and treatment play a crucial role in modernizing TCM diagnosis and treatment. The key of promoting the intelligent development of TCM diagnosis and treatment is to integrate TCM knowledge graph information and build an intelligent TCM-assisted diagnostic and treatment decision-making system with a focus on differential diagnosis and treatment to provide decision-making assistance for TCM clinical diagnosis and treatment.

Currently, due to the vast and complex nature of the TCM knowledge system, which is scattered across numerous ancient texts and literature, existing TCM prescription recommendations cannot effectively handle loose and unstructured knowledge, resulting in low utilization of TCM knowledge. TCM emphasizes a holistic approach, considering various factors such as the body, age, gender, and medical history of the patent, rather than simply treating a single symptom or disease. However, current TCM prescription recommendations lack consideration for personalization. Additionally, since traditional Chinese herbs have certain side effects, it is important to make a proper combination of herbs during formulation of herb prescriptions to achieve a balance in medicine effects and prevent the medicine effects from canceling each other or generating side effects. However, existing TCM prescription recommendations have limitations in handling herbal medicine compatibility, leading to various problems.

### SUMMARY

To address the issues of low utilization of existing TCM prescription recommendation knowledge, inadequate consideration of personalization for patients, and ineffective handling of herbal medicine compatibility, the present disclosure provides a knowledge graph-based method for recommending TCM prescriptions.

To solve the foregoing technical problems, the present disclosure adopts the following technical solution: a knowledge graph-based method for recommending TCM prescriptions, including the following acts:
act 1: collecting TCM data and preprocessing the data to remove duplicate data and standardize entity names;
act 2: performing named entity recognition and relation extraction on the preprocessed TCM data to obtain an entity set E and a relation set R, and constructing a TCM knowledge graph by using entity elements in the entity set E as nodes and relation elements in the relation set R as connection lines between the nodes;
act 3: performing representation learning for the TCM knowledge graph by using a ComplEx model, selecting all symptom nodes and all Chinese materia medica nodes in the TCM knowledge graph, selecting age information nodes, gender information nodes, efficacy information nodes, medicine property information nodes, syndrome information nodes, and treatment and principle nodes that have connection lines with the symptom nodes or Chinese materia medica nodes, representing the selected nodes as complex vectors, and then separately calculating symptom node vector representations s′ fused with other information and Chinese materia medica node vector representations h′ fused with other information;

$${s}^{′} = s + {W}_{a} \cdot a + {W}_{g} \cdot g + {W}_{tr} \cdot tr$$

$${h}^{′} = h + {W}_{ef} \cdot ef + {W}_{pr} \cdot p + {W}_{sy} \cdot sy + {W}_{tr} \cdot tr ;$$

where s denotes a symptom node vector representation; h denotes a Chinese materia medica node vector representation; a denotes an age vector representation; Wₐ denotes an age weight matrix; g denotes a gender vector representation; W_{g} denotes a gender weight matrix; tr denotes a treatment and principle vector representation; Wₜᵣ denotes a treatment method and principle weight matrix; ef denotes an efficacy vector representation; W_{ef} denotes an efficacy weight matrix; p denotes a medicine property vector representation; Wₚᵣ denotes a medicine property weight matrix; sy denotes a syndrome vector representation; and W_{sy} denotes a syndrome weight matrix;
performing tensor-based knowledge graph embedding on the symptom node vector representations s′ fused with other information and the Chinese materia medica node vector representations h′ fused with other information by using an embedding layer of the ComplEx model, mapping the symptom node vector representations s′ fused with other information to a lower-dimensional vector space to obtain symptom node vector representations eₛ′ for which the graph embedding model has been applied, and mapping the Chinese materia medica node vector representations h′ fused with other information to a lower-dimensional vector space to obtain Chinese materia medica node vector representations eₕ′ for which the graph embedding model has been applied;
training the ComplEx model by using a scoring function P(eₛ′, r, eₕ′) of the ComplEx model, and generating a recommendation training set; and
denoting the symptom node vector representations eₛ′ for which the graph embedding model has been applied and the Chinese materia medica node representations for which the graph embedding model has been applied as follows:

$${e}_{s}^{′} = Re ( {e}_{s}^{′} ) + i Im ( {e}_{s}^{′} )$$

$${e}_{h}^{′} = Re ( {e}_{h}^{′} ) + i Im ( {e}_{h}^{′} ) ;$$

where Re(eₛ′) is a real part of eₛ′, Re(eₕ′) is a real part of eₕ′, Im(eₛ′) is an imaginary part of eₛ′, and Im(eₕ′) is an imaginary part of eₕ′.
the scoring function P(eₛ′, r, eₕ′) is expressed by the following formula:

$$P ( {e}_{s}^{′} , r , {e}_{h}^{′} ) = σRe 〈 {e}_{s}^{′} , r , {{\overline{e}}_{h}}^{′} 〉 ;$$

$$Re 〈 {e}_{s}^{′} , r , {{\overline{e}}_{h}}^{′} 〉 = Re ( {\sum }_{k = 1}^{K} {{e}_{s}}_{k}^{′} {r}_{k} {\overline{e}}_{hk}^{′} ) = 〈 Re ( {e}_{s}^{′} ) , Re ( r ) , Re ( {{e}_{h}}^{′} ) 〉 + 〈 Re ( {e}_{s}^{′} ) , Im ( r ) , Im ( {{e}_{h}}^{′} ) 〉 + 〈 Im ( {e}_{s}^{′} ) , Re ( r ) , Im ( {{e}_{h}}^{′} ) 〉 - 〈 Im ( {e}_{s}^{′} ) , Im ( r ) , Re ( {{e}_{h}}^{′} ) 〉 ;$$

where r is a relation vector representation between eₛ′ and eₕ′, σ is an activation function, Re(r) is a real part of r, and Im(r) is an imaginary part of r;
act 4: based on an entity coverage of knowledge graph (KG) in the recommendation training set, freezing or fine-tuning entity embedding vectors learned through training the ComplEx model;

$$f ( {e}_{h , t} ) = \{ \begin{matrix}Freeze ( {v}_{{e}_{h , t}} ) , & if coverage ( {e}_{h , t} ) < threshold \\ Fine - tune ( {v}_{{e}_{h , t}} ) , & otherwise\end{matrix} ;$$

where Freeze(vₑ_{_{h,t}}) represents freezing an entity embedding vector Vₑ_{_{h,t}}, Fine-tune(vₑ_{_{h,t}}) represents fine-tuning an entity embedding vector Vₑ_{_{h,t}}, and threshold represents a determining threshold for the entity coverage;
act 5: learning feature information rₛ of the symptom nodes and feature information rₕ of the Chinese materia medica nodes by using a graph convolutional neural network, where the feature information rₛ of the symptom nodes is obtained through vector representations of the symptom nodes at each layer in the graph convolutional neural network, and the feature information rₕ of the Chinese materia medica nodes is obtained through vector representations of the Chinese materia medica nodes at each layer in the graph convolutional neural network;
where for a symptom node s, a set of one-hop neighbor Chinese materia medica nodes thereof is denoted as Nₛ, and a message from neighbor nodes in the k-th layer is as follows:

$${r}_{{N}_{s}}^{k - 1} = tanh ( {AGGREGATE}_{MEAN} ( \{ {q}_{h \to s}^{k - 1} \} ) ) = tanh ( \frac{1}{\vert {N}_{s} \vert} {\sum }_{h ∈ {N}_{s}} {q}_{h \to s}^{k - 1} ) ;$$

where a vector representation of the symptom node s at the k-th layer in the graph convolutional neural network is denoted as follows:

$${r}_{s}^{k} = tanh ( {W}_{s}^{k} \cdot CONCAT ( {r}_{s}^{k - 1} , {r}_{{N}_{s}}^{k - 1} ) + {b}_{s}^{k} ) ;$$

where |Nₛ| represents a number of adjacent nodes for the symptom node, Wₛ^{k} represents a weight matrix for the symptom node at the k-th layer, bₛ^{k} represents a bias term, tanh represents an activation function, CONCAT represents a vector concatenation operation, and q_{h→s}^{k-1} represents information transmitted from Chinese materia medica nodes in the (k-1)-th layer to the symptom node;
for a Chinese materia medica node h, a set of one-hop neighbor symptom nodes thereof is denoted as Nₕ, and a message from neighbor nodes in the k-th layer is as follows:

$${r}_{{N}_{h}}^{k - 1} = tanh ( {AGGREGATE}_{MEAN} ( \{ {q}_{s \to h}^{k - 1} \} ) ) = tanh ( \frac{1}{\vert {N}_{h} \vert} {\sum }_{s ∈ {N}_{h}} {q}_{s \to h}^{k - 1} ) ;$$

where a vector representation for the Chinese materia medica node h at the k-th layer in the graph convolutional neural network is denoted as follows:

$${r}_{h}^{k} = tanh ( {W}_{h}^{k} \cdot CONCAT ( {r}_{h}^{k - 1} , {r}_{{N}_{s}}^{k - 1} ) + {b}_{h}^{k} ) ;$$

where |Nₕ| represents a number of adjacent nodes for the Chinese materia medica node, Wₕ^{k} represents a weight matrix for the Chinese materia medica node at the k-th layer, bₕ^{k} represents a bias term, tanh represents the activation function, CONCAT represents the vector concatenation operation, and q_{s→h}^{k-1} represents information transmitted from symptom nodes in the (k-1)-th layer to the Chinese materia medica node;
act 6: combining graph features with a recommendation system by using an attention mechanism, and calculating a Query matrix, a Key matrix, and a Value matrix for the symptom nodes according to the symptom node vector representations eₛ′ for which the graph embedding model has been applied and the feature information rₛ of the symptom nodes;

$${Q}_{s} = {W}_{Q}^{( s )} {e}_{s}^{′} ; {K}_{s} = {W}_{K}^{( s )} {r}_{s} ; {V}_{s} = {W}_{V}^{( s )} {e}_{s}^{′} ;$$

calculating a Query matrix, a Key matrix, and a Value matrix for the Chinese materia medica nodes according to the Chinese materia medica node vector representations eₕ′ for which the graph embedding model has been applied and the feature information rₕ of the Chinese materia medica nodes:

$${Q}_{h} = {W}_{Q}^{( h )} {e}_{h}^{′} ; {K}_{h} = {W}_{K}^{( h )} {r}_{h} ; {V}_{h} = {W}_{V}^{( h )} {e}_{h}^{′} ;$$

where W_{Q}^{(s)}, W_{K}^{(s)}, W_{V}^{(s)}, W_{Q}^{(h)}, W_{K}^{(h)}, W_{V}^{(h)} are parameter matrices learned from three linear transformation layers in a multi-head attention layer;
calculating an attention matrix Aₛ for the symptom nodes and an attention matrix Aₕ for the Chinese materia medica nodes by using a softmax function:

$$\begin{matrix}{A}_{s} = softmax ( \frac{{Q}_{s} {K}_{s}^{T}}{\sqrt{{d}_{k}}} ) \\ {A}_{h} = softmax ( \frac{{Q}_{h} {K}_{h}^{T}}{\sqrt{{d}_{k}}} )\end{matrix}$$

where dₖ is a dimension value; and
calculating fused representation vectors s of the symptom nodes and fused representation vectors eₕ* of the Chinese materia medica nodes:

eₛ*=AₛVₛ

eₕ*=AₕVₕ;
act 7: collecting a set of symptom entities sc from a target patient and constructing a multi-hot vector x_{sc}:

$${x}_{s c} [ i ] = \{ \begin{matrix}1 , & if i ∈ s c \\ 0 , & otherwise\end{matrix} ,$$

where 1≤i≤K, K represents a number of the symptom nodes in the TCM knowledge graph
calculating an overall symptom matrix Eₛ* based on the fused representation vectors eₛ* of the symptom nodes:

$${E}_{s}^{*} = [ \begin{matrix}{e}_{s 1}^{*} \\ {e}_{{s}_{2}}^{*} \\ ⋮ \\ {e}_{{s}_{k}}^{*}\end{matrix} ] ,$$

extracting information from the overall symptom matrix Eₛ* by using the multi-hot vector x_{sc} as a mask, to obtain an identified syndrome matrix M_{sc}.

$${M}_{s c} = {E}_{s}^{*} \cdot diag ( {x}_{s c} ) ,$$

where the multi-hot vector x_{sc} is transformed into a diagonal matrix through a diag function, and non-zero rows in the identified syndrome matrix M_{sc} correspond to the fused representation vectors eₛ* of symptom nodes in the set of symptom entities sc;
performing single induction on the identified syndrome matrix M_{sc} by using an average pooling operation, to obtain single representation vectors e_{sc}:

$${e}_{s c} = \frac{1}{k} {\sum }_{i = 1}^{k} {e}_{{s}_{i}}^{*} ;$$

inputting the single representation vectors e_{sc} into the multi-layer perceptron for syndrome induction to obtain final syndrome representations:
where the expression of the multi-layer perceptron is shown as follows:

$$\begin{matrix}{h}_{1} = R e L U ( {W}_{1} {e}_{sc} + {b}_{1} ) \\ {h}_{2} = R e L U ( {W}_{2} {h}_{1} + {b}_{2} ) \\ … \\ {h}_{L} = R e L U ( {W}_{L} {h}_{L - 1} + {b}_{L} ) ;\end{matrix}$$

where W_{L} represents a weight matrix of the L-th layer, b_{L} represents a bias term of the L-th layer, and ReLU represents a non-linear activation function; and
using an output of the multi-layer perceptron as final syndrome representation vectors e_{z}, that is, e_{z}=h_{L};
act 8: calculating an overall Chinese materia medica matrix E_{H}* based on the fused representation vectors eₕ* of the Chinese materia medica nodes:

$${E}_{H}^{*} = [ \begin{matrix}{e}_{{h}_{1}}^{*} \\ {e}_{{h}_{2}}^{*} \\ ⋮ \\ {e}_{{h}_{M}}^{*}\end{matrix} ] ;$$

wherein M represents a total number of candidate Chinese materia medica nodes, the candidate Chinese materia medica nodes are Chinese materia medica nodes in the TCM knowledge graph which have connection lines with symptom nodes in symptom entities in the set sc of symptom entities;
calculating prediction probability vectors m(sc) based on the final syndrome representation vectors e_{z} and the overall Chinese materia medica matrix E_{H}*.
m(sc)=σ(E_{H}*e_{z}^{T}), where σ is an activation function;
for each candidate Chinese materia medica, calculating a binary cross-entropy loss between the prediction probability and a true label, and summing the losses for all Chinese materia medicas; for the set of symptom entities sc, obtaining a prediction probability vector based on m(sc) denoted as ŷ=[ŷ₁, ŷ₂, . . . , ŷ_{M}]^{T}, where each element in the prediction probability vector represents a prediction probability of a corresponding candidate Chinese materia medica node, the TCM recommending device outputs candidate Chinese materia medica nodes each with a prediction probability greater than a predetermined value as recommended Chinese materia medica prescription for the set of symptom entities Sc.

Preferably, in act 1, both structured and unstructured data are collected; the structured data includes TCM dictionaries, databases, and ontologies, while the unstructured data includes TCM literature, clinical records, and expert knowledge.

With the above technical solutions, the present disclosure achieves the following beneficial effects:

The present disclosure applies a knowledge graph embedding model, a multi-head attention mechanism, graph convolutions, and other techniques to combine graph features with a recommendation system. It comprehensively considers patient conditions for TCM prescription recommendations. Taking TCM case description texts as the research object and integrating with TCM knowledge graph information, the present disclosure draws from the clinical experience of renowned TCM experts and fully considers various individual factors such as medicinal properties, efficacy, medical conditions, and patient constitutions. Based on the holistic principles of TCM and the ideology of syndrome differentiation and treatment, the method selects different herbal combinations based on varying symptoms and medical conditions of patients. The proposed TCM prescription recommendation method, incorporating the knowledge graph, considers the complex relationships between individual signs as well as symptoms and medications. It analyzes specific patterns in each prescription comprehensively, providing doctors and patients with more reliable prescription recommendations. The present disclosure assists in clinical decision-making for TCM diagnosis and treatment.

### BRIEF DESCRIPTION OF THE DRAWINGS

FIG. 1 is a flow chart of a knowledge graph-based method for recommending TCM prescriptions according to the present publication.

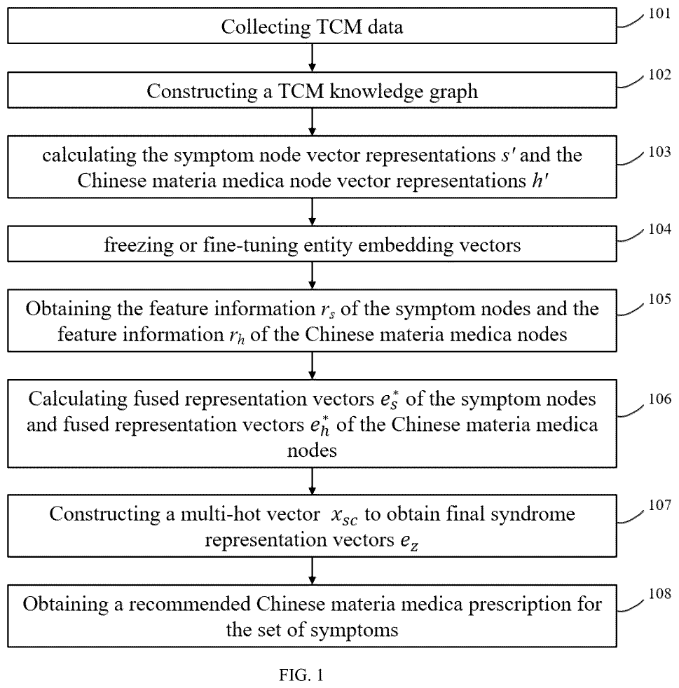

FIG. 2 is a schematic block diagram of a computer that can be used for implementing the method and the system according to the embodiments of the present disclosure.

### DETAILED DESCRIPTION OF THE EMBODIMENTS

### Embodiment 1

The present disclosure relates to a knowledge graph-based method and system for recommending information. The method includes the following acts 101 to 108:

In act 101, associated data for specific applications are collected and preprocessed to remove duplicate data and standardize entity names.

In act 102, the preprocessed associated data are subjected to named entity recognition and relation extraction to obtain an entity set E and a relation set R, and construct a knowledge graph by using entity elements in the entity set E as nodes and relation elements in the relation set R as connection lines between the nodes.

In act 103, representation learning is performed for the knowledge graph by using a ComplEx model. All head information nodes and all tail information nodes are selected in the knowledge graph, and head auxiliary information nodes that have connection lines with the head information nodes and tail auxiliary information nodes that have connection lines with the tail information nodes are selected. The selected nodes are represented as complex vectors, and then head information node vector representations s′ fused with other information and tail information node vector representations h′ fused with other information are separately calculated.

$$\begin{matrix}{s}^{′} = s + {W}_{a 1} \times a 1 + {W}_{a 2} \times a 2 + … + {W}_{a i} \times ai \\ {h}^{′} = s + {W}_{b 1} \times b 1 + {W}_{b 2} \times b 2 + … + {W}_{b j} \times b j\end{matrix}$$

where s denotes a head information node vector representation; h denotes a tail information node vector representation; a1 denotes an first head auxiliary information vector representation; Wₐ₁ denotes an first head auxiliary information weight matrix; a2 denotes a second head auxiliary information vector representation; Wₐ₂ denotes a second head auxiliary information weight matrix; ai denotes a i-th head auxiliary information vector representation; Wₐᵢ denotes a i-th head auxiliary information weight matrix; b1 denotes an first tail auxiliary information vector representation; W_{b1} denotes an first tail auxiliary information weight matrix; b2 denotes a second tail auxiliary information vector representation; W_{b2} denotes a second tail auxiliary information weight matrix; bj denotes a i-th tail auxiliary information vector representation; and W_{bj}, denotes a i-th tail auxiliary information weight matrix, where i and j are both natural numbers.

Tensor-based knowledge graph embedding is performed on the head information node vector representations s′ fused with auxiliary information and the tail information node vector representations h′ fused with auxiliary information by using an embedding layer of the ComplEx model. The head information node vector representations s′ fused with other information are mapped to a lower-dimensional vector space to obtain head information node vector representations eₛ′ for which the graph embedding model has been applied, and the tail information node vector representations h′ fused with other information are mapped to a lower-dimensional vector space to obtain tail information node vector representations eₕ′ for which the graph embedding model has been applied.

The ComplEx model is trained by using a scoring function P(eₛ′, r, eₕ′) of the ComplEx model, and a recommendation training set is generated.

The head information node vector representations eₛ′ for which the graph embedding model has been applied and the tail information node representations for which the graph embedding model has been applied are denoted as follows:

$$\begin{matrix}{e}_{s}^{′} = Re ( {e}_{s}^{′} ) + i Im ( {e}_{s}^{′} ) \\ {e}_{h}^{′} = Re ( {e}_{h}^{′} ) + i Im ( {e}_{h}^{′} ) ;\end{matrix}$$

where Re(eₛ′) is a real part of eₛ′, Re(eₕ′) is a real part of eₕ′, Im(eₛ′) is an imaginary part of eₛ′, and Im(eₕ′) is an imaginary part of eₕ′.

The scoring function P(eₛ′, r, eₕ′) is expressed by the following formula:

$$P ( {e}_{s}^{′} , r , {e}_{h}^{′} ) = σ Re 〈 {e}_{s}^{′} , r , {\overline{e}}_{h}^{′} 〉 ;$$

$$\begin{matrix}Re 〈 {e}_{s}^{′} , r , {\overline{e}}_{h}^{′} 〉 = Re ( {\sum }_{k = 1}^{K} {e}_{{s}_{k}}^{′} {r}_{k} {\overline{e}}_{h k}^{′} ) \\ = 〈 Re ( {e}_{s}^{′} ) , Re ( r ) , Re ( {e}_{h}^{′} ) 〉 + 〈 Re ( {e}_{s}^{′} ) , Im ( r ) , Im ( {e}_{h}^{′} ) 〉 + 〈 Im ( {e}_{s}^{′} ) , Re ( r ) , Im ( {e}_{h}^{′} ) 〉 - 〈 Im ( {e}_{s}^{′} ) , Im ( r ) , Re ( {e}_{h}^{′} ) 〉 ;\end{matrix}$$

where r is a relation vector representation between eₛ′ and eₕ′, σ is an activation function, Re(r) is a real part of r, and Im(r) is an imaginary part of r.

In act 104, based on coverage of knowledge graph (KG) entities in the recommendation training set, entity embedding vectors learned through training the ComplEx model are frozen or fine-tuned.

$$f ( {e}_{h , t} ) = \{ \begin{matrix}Freeze ( {v}_{{e}_{h , t}} ) , & if coverage ( {e}_{h , t} ) < thre s h o l d \\ Fine - tune ( {v}_{{e}_{h , t}} ) , & otherwise\end{matrix} \begin{matrix} \\ \end{matrix} ;$$

where Freeze(vₑ_{_{h,t}}) represents freezing an entity embedding vector Vₑ_{_{h,t}}, Fine-tune(vₑ_{_{h,t}}) represents fine-tuning an entity embedding vector Vₑ_{_{h,t}}, and threshold represents a determining threshold for the entity coverage;

In act 105, feature information of the head information nodes and feature information of the tail information nodes are learnt by using a graph convolutional neural network, where the feature information of the head information nodes is obtained through vector representations of the head information nodes at each layer in the graph convolutional neural network, and the feature information of the tail information nodes is obtained through vector representations of the tail information nodes at each layer in the graph convolutional neural network.

For a head information node s, a set of one-hop neighbor tail information nodes thereof is denoted as Nₛ, and a message from neighbor nodes in the k-th layer is as follows:

$${r}_{{N}_{s}}^{k - 1} = tanh ( {AGGREGATE}_{M E A N} ( \{ {q}_{h \to s}^{k - 1} \} ) ) = tanh ( \frac{1}{\vert {N}_{s} \vert} {\sum }_{h ∈ {N}_{s}} {q}_{h \to s}^{k - 1} ) ;$$

where a vector representation of a head information node s at the k-th layer in the graph convolutional neural network is denoted as follows:

$${r}_{s}^{k} = tanh ( {W}_{s}^{k} \cdot CONCAT ( {r}_{s}^{k - 1} , {r}_{{N}_{s}}^{k - 1} ) + {b}_{s}^{k} ) ;$$

where |Nₛ| represents the number of adjacent nodes for the head information node, Wₛ^{k} represents a weight matrix for the tail information node at the k-th layer, bₛ^{k} represents a bias term, tanh represents an activation function, CONCAT represents a vector concatenation operation, and q_{h→s}^{k-1} represents information transmitted from tail information nodes in the (k-1)-th layer to the head information node.

For a tail information node h, a set of one-hop neighbor head information nodes thereof is denoted as Nₕ, and a message from neighbor nodes in the k-th layer is as follows:

$${r}_{{N}_{h}}^{k - 1} = tanh ( {AGGREGATE}_{MEAN} ( \{ {q}_{s \to h}^{k - 1} \} ) ) = tanh ( \frac{1}{\vert {N}_{h} \vert} {\sum }_{s ∈ {N}_{h}} {q}_{s \to h}^{k - 1} ) ;$$

where a vector representation for the tail information node h at the k-th layer in the graph convolutional neural network is denoted as follows:

$${r}_{h}^{k} = tanh ( {W}_{h}^{k} \cdot CONCAT ( {r}_{h}^{k - 1} , {r}_{{N}_{s}}^{k - 1} ) + {b}_{h}^{k} ) ;$$

where |Nₕ| represents the number of adjacent nodes for the tail information node, Wₕ^{k} represents a weight matrix for the tail information node at the k-th layer, bₕ^{k} represents a bias term, tanh represents an activation function, CONCAT represents a vector concatenation operation, and q_{s→h}^{k-1} represents information transmitted from head information nodes in the (k-1)-th layer to the tail information node.

In act 106, the graph features are combined with a recommendation system by using an attention mechanism, and a Query matrix, a Key matrix, and a Value matrix for the head information nodes are calculated according to the head information node vector representations for which the graph embedding model has been applied and the feature information of the head information nodes:

$${Q}_{s} = {W}_{Q}^{( s )} {e}_{s}^{′} ; {K}_{s} = {W}_{K}^{( s )} {r}_{s} ; {V}_{s} = {W}_{V}^{( s )} {e}_{s}^{′} .$$

A Query matrix, a Key matrix, and a Value matrix for the tail information nodes are calculated according to the tail information node vector representations eₕ′ for which the graph embedding model has been applied and the feature information rₕ of the tail information nodes:

$${Q}_{h} = {W}_{Q}^{( h )} {e}_{h}^{′} ; {K}_{h} = {W}_{K}^{( h )} {r}_{h} ; {V}_{h} = {W}_{V}^{( h )} {e}_{h}^{′} .$$

W_{Q}^{(s)}, W_{K}^{(s)}, W_{V}^{(s)}, W_{Q}^{(h)}, W_{K}^{(h)}, W_{V}^{(h)} are parameter matrices learned from three linear transformation layers in a multi-head attention layer.

An attention matrix Aₛ for the head information nodes and an attention matrix Aₕ for the tail information nodes are calculated by using a softmax function:

$${A}_{s} = softmax ( \frac{{Q}_{s} {K}_{s}^{T}}{\sqrt{{d}_{k}}} )$$

$${A}_{h} = softmax ( \frac{{Q}_{h} {K}_{h}^{T}}{\sqrt{{d}_{k}}} ) ,$$

where dₖ is a dimension value.

Fused representation vectors eₛ* of the head information nodes and fused representation vectors eₕ* of the tail information nodes are calculated:

eₛ*=AₛVₛ

eₕ*=AₕVₕ.

In act 107, a multi-hot vector x_{sc} is construct based on a set of head information entities to be identified sc.

$${x}_{sc} [ i ] = \{ \begin{matrix}1 , & if i ∈ sc \\ 0 , & otherwise\end{matrix} .$$

An overall head information matrix Eₛ* is calculated based on the fused representation vectors s of the head information nodes:

$${E}_{s}^{*} = [ \begin{matrix}{e}_{s 1}^{*} \\ {e}_{{s}_{2}}^{*} \\ ⋮ \\ {e}_{{s}_{k}}^{*}\end{matrix} ] .$$

Information is extracted from the overall head information matrix Eₛ* by using the multi-hot vector x_{sc} as a mask, to obtain an head information characterization matrix M_{sc}:

$${M}_{sc} = {E}_{s}^{*} \cdot diag ( {x}_{sc} ) ,$$

where the multi-hot vector x_{sc} is transformed into a diagonal matrix through a diag function, and non-zero rows in the head information characterization matrix M_{sc} correspond to the fused representation vectors eₛ* of head information nodes in the set of head information entities sc.

Single induction is performed on the head information characterization matrix M_{sc} by using an average pooling operation, to obtain single representation vectors e_{sc}:

$${e}_{sc} = \frac{1}{k} {\sum }_{i = 1}^{k} {e}_{{s}_{i}}^{*} .$$

The single representation vectors e_{sc} are inputted into the multi-layer perceptron for head information characterization induction to obtain final head information characterization.

The expression of the multi-layer perceptron is shown as follows:

$${h}_{1} = Re LU ( {W}_{1} {e}_{sc} + {b}_{1} )$$

$${h}_{2} = Re LU ( {W}_{2} {h}_{1} + {b}_{2} )$$

$$…$$

$${h}_{L} = Re LU ( {W}_{L} {h}_{L - 1} + {b}_{L} ) ;$$

where W_{L} represents a weight matrix of the L-th layer, b_{L} represents a bias term of the L-th layer, and ReLU represents a non-linear activation function.

An output of the multi-layer perceptron is used as final head information characterization vectors e_{z}, that is, e_{z}=h_{L}.

In act 108, an overall tail information matrix E_{H}* is calculated based on the fused representation vectors eₕ* of the tail information nodes:

$${E}_{H}^{*} = [ \begin{matrix}{e}_{{h}_{1}}^{*} \\ {e}_{{h}_{2}}^{*} \\ ⋮ \\ {e}_{{h}_{M}}^{*}\end{matrix} ] .$$

wherein M represents a total number of candidate tail information nodes, the candidate tail information nodes are tail information nodes in the knowledge graph which have connection lines with head information nodes in head information entities in the set sc of head information entities

Prediction probability vectors m(sc) are calculated based on the final head information characterization vectors e_{z} and the overall tail information matrix E_{H}*.
m(sc)=σ(E_{H}*e_{z}^{T}), where σ is an activation function.

For each candidate tail information, a binary cross-entropy loss between the prediction probability and a true label is calculated, and the losses for all tail information are summed; for the set of head information entities sc, a prediction probability vector is obtained based on m(sc) the prediction probability vector is denoted as ŷ=[ŷ₁, ŷ₂, . . . , ŷ_{M}]^{T}, where each element in the prediction probability vector represents a prediction probability of a corresponding candidate tail information node, the model outputs candidate tail information nodes each with a prediction probability greater than a predetermined value as recommended information for the set of head information entities sc.

In addition, knowledge graph-based system for recommending information include data processing and knowledge graph construction module, knowledge graph feature extraction module, multi head attention mechanism feature fusion module, and recommendation module implemented by the computer. Among them, the data processing and knowledge graph construction module is run by the computer to perform acts 101 to 102 described above. The knowledge graph feature extraction module is run by a computer to perform acts 103 to 105 described above. The multi head attention mechanism feature fusion module is run by a computer to perform act 106 describe above. And the recommendation module is run by a computer to perform acts 107 to 108.

In Example 1, by constructing a knowledge graph, a large amount of unstructured and structured data can be integrated into a coherent knowledge network, improving the availability, retrievability, and comprehensibility of entities. The system is therefore enabled to effectively organize and utilize the complex relationships between entities.

In addition, this application integrates knowledge graph and graph neural network (GNN) architecture. Specifically, a graph neural network architecture based on the ComplEx model was designed, which combines representation learning of knowledge graph and graph neural network (GNN) technology. It not only learns low dimensional complex vector representations of entities and relationships, but also innovatively integrates different types of auxiliary information, and dynamically assigns the importance of different types of information using type weight matrices, which enhances the model's consideration of object differences and requirements. In addition, this fusion improves the ability of graph neural network structures to capture and understand associations of high dimensions semantic between different entities, which helps to improve the accuracy of recommended information.

Adopting a multi head attention mechanism to integrate the semantic associations between head and tail nodes in the knowledge graph, dynamically assigning node importance, ensuring that the interaction between different head nodes and the synergies of tail nodes are considered in the information recommendation process, improving the accuracy and interpretability of the recommendation system.

Using a multi-layer perceptron to characterize the nonlinear interaction relationship between head node sets, and extracting the overall head information representation from the fusion representation of multiple head nodes, in order to accurately predict the correspondence between head information and head information representation, providing a more accurate basis for personalized information recommendation.

According to the disclosed method, both knowledge graph representation learning and information recommendation tasks were optimized during the training process. The performance of the model in both knowledge representation and actual information recommendation was ensured by a joint loss function. The experimental results showed that it outperformed other comparative models in various evaluation indicators, demonstrating high accuracy and practicality.

### Embodiment 2

Given the advantages of the method in Embodiment 1 in information recommendation, it is particularly suitable for recommending TCM prescriptions.

A knowledge graph-based method for recommending TCM prescriptions includes the following acts 101 to 108.

In act 101, TCM data are collected and preprocessed to remove duplicate data and standardize entity names. Both structured and unstructured data are collected. The structured data includes TCM dictionaries, databases, and ontologies, while the unstructured data includes TCM literature, clinical records, and expert knowledge.

In act 102, the preprocessed TCM data are subjected to named entity recognition and relation extraction to obtain an entity set E and a relation set R, and construct a TCM knowledge graph by using entity elements in the entity set E as nodes and relation elements in the relation set R as connection lines between the nodes.

In act 103, representation learning is performed for the TCM knowledge graph by using a ComplEx model. All symptom nodes and all Chinese materia medica nodes are selected in the TCM knowledge graph, and age information nodes, gender information nodes, efficacy information nodes, medicine property information nodes, syndrome information nodes, and treatment and principle nodes that have connection lines with the symptom nodes or Chinese materia medica nodes are selected. The selected nodes are represented as complex vectors, and then symptom node vector representations s′ fused with other information and Chinese materia medica node vector representations h′ fused with other information are separately calculated.

$${s}^{′} = s + {W}_{a} \cdot a + {W}_{g} \cdot g + {W}_{tr} \cdot tr$$

$${h}^{′} = h + {W}_{ef} \cdot ef + {W}_{pr} \cdot p + {W}_{sy} \cdot sy + {W}_{tr} \cdot tr ;$$

where s denotes a symptom node vector representation; h denotes a Chinese materia medica node vector representation; a denotes an age vector representation; Wₐ denotes an age weight matrix; g denotes a gender vector representation; W_{g} denotes a gender weight matrix; tr denotes a treatment and principle vector representation; Wₜᵣ denotes a treatment and principle weight matrix; ef denotes an efficacy vector representation; W_{ef} denotes an efficacy weight matrix; p denotes a medicine property vector representation; Wₚᵣ denotes a medicine property weight matrix; sy denotes a syndrome vector representation; and W_{sy} denotes a syndrome weight matrix.

Tensor-based knowledge graph embedding is performed on the symptom node vector representations s′ fused with other information and the Chinese materia medica node vector representations h′ fused with other information by using an embedding layer of the ComplEx model. The symptom node vector representations s′ fused with other information are mapped to a lower-dimensional vector space to obtain symptom node vector representations eₛ′ for which the graph embedding model has been applied, and the Chinese materia medica node vector representations h′ fused with other information are mapped to a lower-dimensional vector space to obtain Chinese materia medica node vector representations eₕ′ for which the graph embedding model has been applied.

The ComplEx model is trained by using a scoring function P(eₛ′, r, eₕ′) of the ComplEx model, and a recommendation training set is generated.

The symptom node vector representations eₛ′ for which the graph embedding model has been applied and the Chinese materia medica node representations for which the graph embedding model has been applied are denoted as follows:

$${e}_{s}^{′} = Re ( {e}_{s}^{′} ) + i Im ( {e}_{s}^{′} )$$

$${e}_{h}^{′} = Re ( {e}_{h}^{′} ) + i Im ( {e}_{h}^{′} ) ;$$

where Re(eₛ′) is a real part of eₛ′, Re(eₕ′) is a real part of eₕ′, Im(eₛ′) is an imaginary part of eₛ′, and Im(eₕ′) is an imaginary part of eₕ′.

The scoring function P(eₛ′, r, eₕ′) is expressed by the following formula:

$$P ( {e}_{s}^{′} , r , {e}_{h}^{′} ) = σRe 〈 {e}_{s}^{′} , r , {{\overline{e}}_{h}}^{′} 〉 ;$$

$$Re 〈 {e}_{s}^{′} , r , {{\overline{e}}_{h}}^{′} 〉 = Re ( {\sum }_{k = 1}^{K} {{e}_{s}}_{k}^{′} {r}_{k} {\overline{e}}_{hk}^{′} ) = 〈 Re ( {e}_{s}^{′} ) , Re ( r ) , Re ( {{e}_{h}}^{′} ) 〉 + 〈 Re ( {e}_{s}^{′} ) , Im ( r ) , Im ( {{e}_{h}}^{′} ) 〉 + 〈 Im ( {e}_{s}^{′} ) , Re ( r ) , Im ( {{e}_{h}}^{′} ) 〉 - 〈 Im ( {e}_{s}^{′} ) , Im ( r ) , Re ( {{e}_{h}}^{′} ) 〉 ;$$

where r is a relation vector representation between eₛ′ and eₕ′, σ is an activation function, Re(r) is a real part of r, and Im(r) is an imaginary part of r.

In act 104, based on coverage of knowledge graph (KG) entities in the recommendation training set, entity embedding vectors learned through training the ComplEx model are frozen or fine-tuned.

$$f ( {e}_{h , t} ) = \{ \begin{matrix}Freeze ( {v}_{{e}_{h , t}} ) , & if coverage ( {e}_{h , t} ) < threshold \\ Fine - tune ( {v}_{{e}_{h , t}} ) , & otherwise\end{matrix} ;$$

where Freeze(vₑ_{_{h,t}}) represents freezing an entity embedding vector Vₑ_{_{h,t}}, Fine-tune(vₑ_{_{h,t}}) represents fine-tuning an entity embedding vector Vₑ_{_{h,t}}, and threshold represents a determining threshold for the entity coverage;

In act 105, feature information of the symptom nodes and feature information of the Chinese materia medica nodes are learnt by using the graph convolutional neural network, where the feature information of the symptom nodes is obtained through vector representations of the symptom nodes at each layer in the graph convolutional neural network, and the feature information of the Chinese materia medica nodes is obtained through vector representations of the Chinese materia medica nodes at each layer in the graph convolutional neural network.

For a symptom node s, a set of one-hop neighbor Chinese materia medica nodes thereof is denoted as Nₛ, and a message from neighbor nodes in the k-th layer is as follows:

$${r}_{{N}_{s}}^{k - 1} = tanh ( {AGGREGATE}_{M E A N} ( \{ {q}_{h \to s}^{k - 1} \} ) ) = tanh ( \frac{1}{\vert {N}_{s} \vert} {\sum }_{h ∈ {N}_{s}} {q}_{h \to s}^{k - 1} ) ;$$

where a vector representation of a symptom node s at the k-th layer in the graph convolutional neural network is denoted as follows:

$${r}_{s}^{k} = tanh ( {W}_{s}^{k} \cdot CONCAT ( {r}_{s}^{k - 1} , {r}_{{N}_{s}}^{k - 1} ) + {b}_{s}^{k} ) ;$$

where |Nₛ| represents the number of adjacent nodes for the symptom node, Wₛ^{k} represents a weight matrix for the symptom node at the k-th layer, bₛ^{k} represents a bias term, tanh represents an activation function, CONCAT represents a vector concatenation operation, and q_{h→s}^{k-1} represents information transmitted from Chinese materia medica nodes in the (k-1)-th layer to the symptom node.

For a Chinese materia medica node h, a set of one-hop neighbor symptom nodes thereof is denoted as Nₕ, and a message from neighbor nodes in the k-th layer is as follows:

$${r}_{{N}_{h}}^{k - 1} = tanh ( {AGGREGATE}_{M E A N} ( \{ {q}_{s \to h}^{k - 1} \} ) ) = tanh ( \frac{1}{\vert {N}_{h} \vert} {\sum }_{s ∈ {N}_{h}} {q}_{s \to h}^{k - 1} ) ;$$

where a vector representation for the Chinese materia medica node h at the k-th layer in the graph convolutional neural network is denoted as follows:

$${r}_{h}^{k} = tanh ( {W}_{h}^{k} \cdot CONCAT ( {r}_{h}^{k - 1} , {r}_{{N}_{s}}^{k - 1} ) + {b}_{h}^{k} ) ;$$

where |Nₕ| represents the number of adjacent nodes for the Chinese materia medica node, Wₕ^{k} represents a weight matrix for the symptom node at the k-th layer, bₕ^{k} represents a bias term, tanh represents an activation function, CONCAT represents a vector concatenation operation, and q_{s→h}^{k-1} represents information transmitted from symptom nodes in the (k-1)-th layer to the Chinese materia medica node.

In act 106, the graph features are combined with a recommendation system by using the attention mechanism model, and a Query matrix, a Key matrix, and a Value matrix for the symptom nodes are calculated according to the symptom node vector representations for which the graph embedding model has been applied and the feature information of the symptom nodes:

$${Q}_{s} = {W}_{Q}^{( s )} {e}_{s}^{′} ; {K}_{s} = {W}_{K}^{( s )} {r}_{s} ; {V}_{s} = {W}_{V}^{( s )} {e}_{s}^{′} .$$

A Query matrix, a Key matrix, and a Value matrix for the Chinese materia medica nodes are calculated according to the Chinese materia medica node vector representations eₕ′ for which the graph embedding model has been applied and the feature information rₕ of the Chinese materia medica nodes:

$${Q}_{h} = {W}_{Q}^{( h )} {e}_{h}^{′} ; {K}_{h} = {W}_{K}^{( h )} {r}_{h} {V}_{h} = {W}_{V}^{( h )} {e}_{h}^{′} .$$

W_{Q}^{(s)}, W_{K}^{(s)}, W_{V}^{(s)}, W_{Q}^{(h)}, W_{K}^{(h)}, W_{V}^{(h)} are parameter matrices learned from three linear transformation layers in a multi-head attention layer.

An attention matrix Aₛ for the symptom nodes and an attention matrix Aₕ for the Chinese materia medica nodes are calculated by using a softmax function:

$$\begin{matrix}{A}_{s} = softmax ( \frac{{Q}_{s} {K}_{s}^{T}}{\sqrt{{d}_{k}}} ) \\ {A}_{h} = softmax ( \frac{{Q}_{h} {K}_{h}^{T}}{\sqrt{{d}_{k}}} ) ,\end{matrix}$$

where dₖ is a dimension value.

Fused representation vectors eₛ* of the symptom nodes and fused representation vectors eₕ′ of the Chinese materia medica nodes are calculated:

e=AₛVₛ

eₕAₕVₕ.

In act 107, a set of symptom entities is collected from a target patient and a multi-hot vector x_{sc} is construct:

$${x}_{s c} [ i ] = \{ \begin{matrix}1 , & if i ∈ s c \\ 0 , & otherwi s e\end{matrix} ,$$

wherein 1≤i≤K, K represents a number of the symptom nodes in the TCM knowledge graph.

An overall symptom matrix Eₛ* is calculated based on the fused representation vectors eₛ* of the symptom nodes:

$${E}_{s}^{*} = [ \begin{matrix}{e}_{s 1}^{*} \\ {e}_{{s}_{2}}^{*} \\ ⋮ \\ {e}_{{s}_{k}}^{*}\end{matrix} ] .$$

Information is extracted from the overall symptom matrix Eₛ* by using the multi-hot vector x_{sc} as a mask, to obtain an identified syndrome matrix M_{sc}:

$${M}_{s c} = {E}_{s}^{*} \cdot diag ( {x}_{s c} ) ,$$

where the multi-hot vector x_{sc} is transformed into a diagonal matrix through a diag function, and non-zero rows in the identified syndrome matrix M_{sc} correspond to the fused representation vectors s of symptom nodes in the set of symptom entities sc.

Single induction is performed on the identified syndrome matrix M_{sc} by using an average pooling operation, to obtain single representation vectors e_{sc}:

$${e}_{𝓈 c} = \frac{1}{k} {\sum }_{i = 1}^{k} {e}_{{𝓈}_{i}}^{*} .$$

The single representation vectors e_{sc} are inputted into the multi-layer perceptron for syndrome induction to obtain final syndrome representations.

The expression of the multi-layer perceptron is shown as follows:

$$\begin{matrix}{h}_{1} = R e L U ( {W}_{1} {e}_{sc} + {b}_{1} ) \\ {h}_{2} = R e L U ( {W}_{2} {h}_{1} + {b}_{2} ) \\ … \\ {h}_{L} = R e L U ( {W}_{L} {h}_{L - 1} + {b}_{L} ) ;\end{matrix}$$

where W_{L} represents a weight matrix of the L-th layer, b_{L} represents a bias term of the L-th layer, and ReLU represents a non-linear activation function.

An output of the multi-layer perceptron is used as final syndrome representation vectors e_{z}, that is, e_{z}=h_{L}.

In act 108, an overall Chinese materia medica matrix E_{H}* is calculated based on the fused representation vectors eₕ* of the Chinese materia medica nodes:

$${E}_{H}^{*} = [ \begin{matrix}{e}_{{h}_{1}}^{*} \\ {e}_{{h}_{2}}^{*} \\ : \\ {e}_{{h}_{M}}^{*}\end{matrix} ] ,$$

wherein M represents a total number of candidate Chinese materia medica nodes, the candidate Chinese materia medica nodes are Chinese materia medica nodes in the TCM knowledge graph which have connection lines with symptom nodes in symptom entities in the set SC of symptom entities.

Prediction probability vectors m(sc) are calculated based on the final syndrome representation vectors e_{z} and the overall Chinese materia medica matrix E_{H}*.

m(sc)=σ(E_{H}*e_{z}^{T}), where σ is an activation function.

For each candidate Chinese materia medica, a binary cross-entropy loss between the prediction probability and a true label is calculated, and the losses for all Chinese materia medicas are summed; for the set of symptom entities sc, a prediction probability vector is obtained based on m(sc) the prediction probability vector is denoted as ŷ=[ŷ₁, ŷ₂, . . . , ŷ_{M}]^{T}, where each element in the prediction probability vector represents a prediction probability of a corresponding candidate Chinese material medical node, the TCM recommending device outputs candidate Chinese materia medica nodes each with a prediction probability greater than a predetermined value as recommended Chinese materia medica prescription for the set of symptom entities sc.

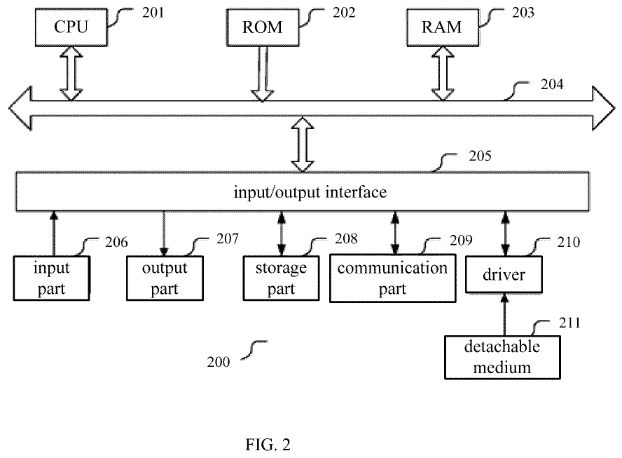

FIG. 2 shows a schematic block diagram of a computer that can be used for implementing the method and the system according to the embodiments of the present disclosure.

In FIG. 2, a central processing unit (CPU) 201 executes various processing according to a program stored in a read-only memory (ROM) 202 or a program loaded from a storage part 208 to a random access memory (RAM) 203. In the RAM 203, data needed at the time of execution of various processing and the like by the CPU 201 is also stored according to requirements. The CPU 201, the ROM 202 and the RAM 203 are connected to each other via a bus 204. An input/output interface 205 is also connected to the bus 204.

The following components are connected to the input/output interface 205: an input part 206 (including a keyboard, a mouse and the like); an output part 207 (including a display, such as a Cathode Ray Tube (CRT), a Liquid Crystal Display (LCD) and the like, as well as a loudspeaker and the like); the storage part 208 (including a hard disc and the like); and a communication part 209 (including a network interface card such as an LAN card, a modem and so on). The communication part 209 performs communication processing via a network such as the Internet. According to requirements, a driver 210 may also be connected to the input/output interface 205. A detachable medium 211 such as a magnetic disc, an optical disc, a magnetic optical disc, a semiconductor memory and the like may be installed on the driver 210 according to requirements, such that a computer program read therefrom is installed in the storage part 208 according to requirements.

In the case of carrying out the foregoing series of processing by software, programs constituting the software are installed from a network such as the Internet or a storage medium such as the detachable medium 211.

Those skilled in the art should appreciate that such a storage medium is not limited to the detachable medium 211 storing therein a program and distributed separately from the apparatus to provide the program to a user as shown in FIG. 2. Examples of the detachable medium 211 include a magnetic disc (including floppy disc (registered trademark)), a compact disc (including compact disc read-only memory (CD-ROM) and digital versatile disc (DVD), a magneto optical disc (including mini disc (MD)(registered trademark)), and a semiconductor memory. Or, the storage medium may be hard discs and the like included in the ROM 202 and the storage part 208 in which programs are stored, and are distributed concurrently with the apparatus including them to users.

The present disclosure further proposes a program product storing therein a machine-readable instruction code that, when read and executed by a machine, can implement the aforesaid method according to the embodiment of the present disclosure.

Correspondingly, a storage medium for carrying the program product storing therein the machine-readable instruction code is also included in the disclosure of the present disclosure. The storage medium includes but is not limited to a floppy disc, an optical disc, a magnetic optical disc, a memory card, a memory stick and the like.

What is claimed is:

1. A knowledge graph-based method for recommending traditional Chinese medicine (TCM) prescriptions, comprising:
collecting TCM data and preprocessing the data to remove duplicate data and standardize entity names;
performing named entity recognition and relation extraction on the preprocessed TCM data to obtain an entity set E and a relation set R, and constructing a TCM knowledge graph by using entity elements in the entity set E as nodes and relation elements in the relation set R as connection lines between the nodes; and
performing following process, by a TCM recommending device comprising a processor and a storage medium that comprises a ComplEx model, a graph convolutional neural network and an attention mechanism model;
performing representation learning for the TCM knowledge graph by using the ComplEx model, selecting all symptom nodes and all Chinese materia medica nodes in the TCM knowledge graph, selecting age information nodes, gender information nodes, efficacy information nodes, medicine property information nodes, syndrome information nodes, and treatment method and principle nodes that have connection lines with the symptom nodes or Chinese materia medica nodes, representing the selected nodes as complex vectors, and then separately calculating symptom node vector representations s′ fused with other information and Chinese materia medica node vector representations h′ fused with other information;
$$\begin{matrix}{𝓈}^{′} = 𝓈 + {W}_{a} \cdot a + {W}_{g} \cdot g + {W}_{t r} \cdot tr \\ {h}^{′} = h + {W}_{e f} \cdot ef + {W}_{p r} \cdot p + {W}_{𝓈 y} \cdot sy + {W}_{t r} \cdot tr ;\end{matrix}$$
wherein s denotes a symptom node vector representation; h denotes a Chinese materia medica node vector representation; a denotes an age vector representation; Wₐ denotes an age weight matrix; g denotes a gender vector representation; W_{g} denotes a gender weight matrix; tr denotes a treatment and principle vector representation; Wₜᵣ denotes a treatment and principle weight matrix; ef denotes an efficacy vector representation; W_{ef} denotes an efficacy weight matrix; p denotes a medicine property vector representation; Wₚᵣ denotes a medicine property weight matrix; sy denotes a syndrome vector representation; and W_{sy} denotes a syndrome weight matrix;
performing tensor-based knowledge graph embedding on the symptom node vector representations s′ fused with other information and the Chinese materia medica node vector representations h′ fused with other information by using an embedding layer of the ComplEx model, mapping the symptom node vector representations s′ fused with other information to a lower-dimensional vector space to obtain symptom node vector representations eₛ′ for which the graph embedding model has been applied, and mapping the Chinese materia medica node vector representations h′ fused with other information to a lower-dimensional vector space to obtain Chinese materia medica node vector representations eₕ′ for which the graph embedding model has been applied, thereby obtaining a recommendation training set which comprises the symptom node vector representations eₛ′ and the Chinese materia medica node vector representations eₕ′ by using the ComplEx model;
based on an entity coverage of knowledge graph (KG) in the recommendation training set, freezing or fine-tuning entity embedding vectors learned through training the ComplEx model;
$$f ( {e}_{h , t} ) = \{ \begin{matrix}Freeze ( {v}_{{e}_{h , t}} ) , & if coverage ( {e}_{h , t} ) < threshold \\ Fine - tune ( {v}_{{e}_{h , t}} ) , & otherwise\end{matrix} \begin{matrix} \\ \end{matrix} ;$$
wherein Freeze(vₑ_{_{h,t}}) represents freezing an entity embedding vector Vₑ_{_{h,t}} Fine-tune(vₑ_{_{h,t}}) represents fine-tuning an entity embedding vector Vₑ_{_{h,t}}, and threshold represents a determining threshold for the entity coverage;
learning feature information rₛ of the symptom nodes and feature information rₕ of the Chinese materia medica nodes by using the graph convolutional neural network, wherein the feature information rₛ of the symptom nodes is obtained through vector representations of the symptom nodes at each layer in the graph convolutional neural network, and the feature information rₕ of the Chinese materia medica nodes is obtained through vector representations of the Chinese materia medica nodes at each layer in the graph convolutional neural network;
for a symptom node s, a set of one-hop neighbor Chinese materia medica nodes thereof is denoted as Nₛ, and a message from neighbor nodes in a k-th layer is as follows:
$${r}_{{N}_{s}}^{k - 1} = tanh ( {AGGREGATE}_{MEAN} ( \{ {q}_{h \to s}^{k - 1} \} ) ) = tanh ( \frac{1}{\vert {N}_{s} \vert} {\sum }_{h ∈ {N}_{s}} {q}_{h \to s}^{k - 1} ) ;$$
wherein a vector representation of the symptom node s at the k-th layer in the graph convolutional neural network is denoted as follows:
$${r}_{s}^{k} = tanh ( {W}_{s}^{k} \cdot CONCAT ( {r}_{s}^{k - 1} , {r}_{{N}_{s}}^{k - 1} ) + {b}_{s}^{k} ) ;$$
wherein |Nₛ| represents a number of adjacent nodes for the symptom node, Wₛ^{k} represents a weight matrix for the symptom node at the k-th layer, bₛ^{k} represents a bias term, tanh represents an activation function, CONCAT represents a vector concatenation operation, and q_{h→s}^{k-1} represents information transmitted from Chinese materia medica nodes in a (k-1)-th layer to the symptom node;
for a Chinese materia medica node h, a set of one-hop neighbor symptom nodes thereof is denoted as Nₕ, and a message from neighbor nodes in the k-th layer is as follows:
$${r}_{{N}_{h}}^{k - 1} = tanh ( {AGGREGATE}_{MEAN} ( \{ {q}_{s \to h}^{k - 1} \} ) ) = tanh ( \frac{1}{\vert {N}_{h} \vert} {\sum }_{s ∈ {N}_{h}} {q}_{s \to h}^{k - 1} ) ;$$
wherein a vector representation for the Chinese materia medica node h at the k-th layer in the graph convolutional neural network is denoted as follows:
$${r}_{h}^{k} = tanh ( {W}_{h}^{k} \cdot CONCAT ( {r}_{h}^{k - 1} , {r}_{{N}_{s}}^{k - 1} ) + {b}_{h}^{k} ) ;$$
wherein |Nₕ| represents a number of adjacent nodes for the Chinese materia medica node, Wₕ^{k} represents a weight matrix for the Chinese materia medica node at the k-th layer, bₕ^{k} represents a bias term, tanh represents the activation function, CONCAT represents the vector concatenation operation, and q_{s→h}^{k-1} represents information transmitted from symptom nodes in the (k-1)-th layer to the Chinese materia medica node;
combining graph features with a recommendation system by using the attention mechanism model, and calculating a Query matrix, a Key matrix, and a Value matrix for the symptom nodes according to the symptom node vector representations eₛ′ for which the graph embedding model has been applied and the feature information rₛ of the symptom nodes:
$${Q}_{s} = {W}_{Q}^{( s )} {e}_{s}^{′} ; {K}_{s} = {W}_{K}^{( s )} {r}_{s} ; {V}_{s} = {W}_{V}^{( s )} {e}_{s}^{′} ;$$
calculating a Query matrix, a Key matrix, and a Value matrix for the Chinese materia medica nodes according to the Chinese materia medica node vector representations eₕ′ for which the graph embedding model has been applied and the feature information rₕ of the Chinese materia medica nodes:
$${Q}_{h} = {W}_{Q}^{( h )} {e}_{h}^{′} ; {K}_{h} = {W}_{K}^{( h )} {r}_{h} ; {V}_{h} = {W}_{V}^{( h )} {e}_{h}^{′} ;$$
wherein W_{Q}^{(s)}, W_{K}^{(s)}, W_{V}^{(s)}, W_{Q}^{(h)}, W_{K}^{(h)}, W_{V}^{(h)} are parameter matrices learned from three linear transformation layers in a multi-head attention layer;
calculating an attention matrix Aₛ for the symptom nodes and an attention matrix Aₕ for the Chinese materia medica nodes by using a softmax function:
$$\begin{matrix}{A}_{s} = softmax ( \frac{{Q}_{s} {K}_{s}^{T}}{\sqrt{{d}_{k}}} ) \\ {A}_{h} = softmax ( \frac{{Q}_{h} {K}_{h}^{T}}{\sqrt{{d}_{k}}} )\end{matrix} ,$$
wherein dₖ is a dimension value; and
calculating fused representation vectors eₛ* of the symptom nodes and fused representation vectors eₕ* of the Chinese materia medica nodes:
eₛ*=AₛVₛ
eₕ*=AₕVₕ;
collecting a set sc of symptom entities from a target patient and constructing a multi-hot vector x_{sc}:
$${x}_{sc} [ i ] = \{ \begin{matrix}1 , & if i ∈ s c \\ 0 , & otherwise\end{matrix} ,$$
wherein 1≤i≤K, K represents a number of the symptom nodes in the TCM knowledge graph;
calculating an overall symptom matrix Eₛ* based on the fused representation vectors eₛ* of the symptom nodes:
$${E}_{s}^{*} = [ \begin{matrix}{e}_{s 1}^{*} \\ {e}_{{s}_{2}}^{*} \\ ⋮ \\ {e}_{{s}_{k}}^{*}\end{matrix} ] ;$$
extracting information from the overall symptom matrix Eₛ* by using the multi-hot vector x_{sc} as a mask, to obtain an identified syndrome matrix M_{sc}:
$${M}_{sc} = {E}_{s}^{*} \cdot diag ( {x}_{sc} ) ,$$
wherein the multi-hot vector x_{sc} is transformed into a diagonal matrix through a diag function, and non-zero rows in the identified syndrome matrix M_{sc} correspond to the fused representation vectors eₛ* of symptom nodes in the set of symptom entities SC;
performing single induction on the identified syndrome matrix M_{sc} by using an average pooling operation, to obtain single representation vectors e_{sc}:
$${e}_{sc} = \frac{1}{k} {\sum }_{i = 1}^{k} {e}_{{s}_{i}}^{*} ;$$
inputting the single representation vectors e_{sc} into the multi-layer perceptron for syndrome induction to obtain final syndrome representations:
wherein the expression of the multi-layer perceptron is shown as follows:
$$\begin{matrix}{h}_{1} = ReLU ( {W}_{1} {e}_{sc} + {b}_{1} ) \\ {h}_{2} = ReLU ( {W}_{2} {h}_{1} + {b}_{2} ) \\ ⋯ \\ {h}_{L} = ReLU ( {W}_{L} {h}_{L - 1} + {b}_{L} )\end{matrix} ;$$
wherein W_{L} represents a weight matrix of a L-th layer, b_{L} represents a bias term of the L-th layer, and ReLU represents a non-linear activation function;
using an output of the multi-layer perceptron as final syndrome representation vectors e_{z}, that is, e_{z}=h_{L};
calculating an overall Chinese materia medica matrix E_{H}* based on the fused representation vectors eₕ* of the Chinese materia medica nodes:
$${E}_{H}^{*} = [ \begin{matrix}{e}_{{h}_{1}}^{*} \\ {e}_{{h}_{2}}^{*} \\ ⋮ \\ {e}_{{h}_{M}}^{*}\end{matrix} ] ,$$
wherein M represents a total number of candidate Chinese materia medica nodes, the candidate Chinese materia medica nodes are Chinese materia medica nodes in the TCM knowledge graph which have connection lines with symptom nodes in symptom entities in the set sc of symptom entities;
calculating prediction probability vectors m(sc) based on the final syndrome representation vectors e_{z} and the overall Chinese materia medica matrix E_{H}*:
m(sc)=σ(E_{H}*e_{z}^{T}), wherein σ is an activation function;
for the set of symptom entities sc, obtaining a prediction probability vector based on m(SC) denoted as ŷ=[ŷ₁, ŷ₂, . . . , ŷ_{M}]^{T}, wherein each element in the prediction probability vector represents a prediction probability of a corresponding candidate Chinese materia medica node, the TCM recommending device outputs candidate Chinese materia medica nodes each with a prediction probability greater than a predetermined value as recommended Chinese materia medica prescription for the set of symptom entities sc.

2. The knowledge graph-based method for recommending TCM prescriptions according to claim 1, wherein in act of the collecting TCM data, both structured and unstructured data are collected; and the structured data comprises TCM dictionaries, databases, and ontologies, while the unstructured data comprises TCM literature, clinical records, and expert knowledge.

3. The knowledge graph-based method for recommending TCM prescriptions according to claim 1, further comprising:
training the ComplEx model by using a scoring function P(eₛ′, r, eₕ′) of the ComplEx model; and
denoting the symptom node vector representations eₛ′ for which the graph embedding model has been applied and the Chinese materia medica node representations for which the graph embedding model has been applied as follows:
$$\begin{matrix}{e}_{s}^{′} = Re ( {e}_{s}^{′} ) + i Im ( {e}_{s}^{′} ) \\ {e}_{h}^{′} = Re ( {e}_{h}^{′} ) + i Im ( {e}_{h}^{′} )\end{matrix} ;$$
wherein Re(eₛ′) is a real part of eₛ′, Re(eₕ′) is a real part of eₕ′, Im(eₛ′) is an imaginary part of eₛ′, and Im(eₕ′) is an imaginary part of eₕ′;
the scoring function P(eₛ′, r, eₕ′) is expressed by following formula:
$$P ( {e}_{s}^{′} , r , {e}_{h}^{′} ) = σ Re 〈 {e}_{s}^{′} , r , {\overline{e}}_{h}^{′} 〉 ;$$
$$Re 〈 {e}_{s}^{′} , r , {\overline{e}}_{h}^{′} 〉 = Re ( {\sum }_{k = 1}^{K} {e}_{{s}_{k}}^{′} {r}_{k} {\overline{e}}_{h k}^{′} ) = 〈 Re ( {e}_{s}^{′} ) , Re ( r ) , Re ( {e}_{h}^{′} ) 〉 + 〈 Re ( {e}_{s}^{′} ) , Im ( r ) , Im ( {e}_{h}^{′} ) 〉 + 〈 Im ( {e}_{s}^{′} ) , Re ( r ) , Im ( {e}_{h}^{′} ) 〉 - 〈 Im ( {e}_{s}^{′} ) , Im ( r ) , Re ( {e}_{h}^{′} ) 〉$$
wherein r is a relation vector representation between eₛ′ and eₕ′, σ is an activation function, Re(r) is a real part of r, and Im(r) is an imaginary part of r;

4. The knowledge graph-based method for recommending TCM prescriptions according to claim 1, wherein for prediction probability vectors m(sc):
m(sc)=σ(E_{H}*e_{z}^{T})
during a training process, for each candidate Chinese materia medica, a binary cross-entropy loss between the prediction probability and a true label is calculated, and the losses for all Chinese materia medicas are summed.
